(19) United States
(12) Patent Application Publication
(10) Pub. No.: US 2025/0268842 A1
(43) Pub. Date: Aug. 28, 2025

(54) Compositions for reducing oxidative stress

(71) Applicants: N.V. Nutricia, Zoetermeer (NL); NUTRICIA EARLY LIFE NUTRITION ( SHANGHAI ) CO., LTD., Shanghai (CN)

(72) Inventors: Ingrid Brunhilde Renes, Utrecht (NL); Gabriël Thomassen, Utrecht (NL); Jan Knol, Utrecht (NL); Thomas Ludwig, Utrecht (NL); Haofang Jin, Shanghai (CN)

(73) Assignees: N.V. Nutricia, Zoetermeer (NL); NUTRICIA EARLY LIFE NUTRITION ( SHANGHAI ) CO., LTD., Shanghai (CN)

(21) Appl. No.: 19/207,464

(22) Filed: May 14, 2025

Related U.S. Application Data
(62) Division of application No. PCT/EP2023/081964, filed on Nov. 15, 2023.

(30) Foreign Application Priority Data
Nov. 17, 2022 (WO) PCT/CN2022/132459

(51) Int. Cl. A61K 31/047; A23L 33/00; A23L 33/18; A61K 31/231; A61K 31/702; A61K 38/17; A61P 29/00

(52) U.S. Cl. CPC A61K 31/047; A23L 33/18; A23L 33/40; A61K 31/231; A61K 31/702; A61K 38/1761; A61P 29/00

(57) ABSTRACT

The present invention relates to compositions comprising lutein, non-digestible carbohydrates and at least one of casein phosphopetides or 1,3-dioleoyl-2-palmitoylglycerol.

### FIELD OF THE INVENTION

The present invention relates to compositions comprising lutein, non-digestible carbohydrates, casein phosphopeptides and/or 1,3-dioleoyl-2-palmitoylglycerol useful in reducing oxidative stress and related disorders in a human subject.

### BACKGROUND OF THE INVENTION

Oxidative stress refers to the imbalance between abundance of reactive oxygen species (ROS) or reactive nitrogen species (RNS) and insufficient antioxidant defence. Injuries caused by ROS/RNS species on biomolecules, cell membranes, enzymes, receptors, leads to cell function alteration, which in turn results in oxidative stress damage and oxidative stress-mediated disorders.

The mechanism behind oxidative stress damage and associated disorders include endogenous and exogenous production of reactive species, as well as the antioxidants system. Hallmarks of such mechanism include DNA damage, cell membrane damage and leakage, apoptosis and inflammation.

Oxidative stress disorders also include several age-related disorders, such as cardiovascular diseases (CVD), chronic obstructive pulmonary disease, chronic kidney disease, neurological and neurodegenerative diseases, cancer, sarcopenia, diabetes, respiratory diseases, rheumatoid arthritis, diseases associated with elevated concentration of low density lipoprotein (LDL), and age-related macular degeneration (AMD). In neurological diseases, reactive products of PUFA peroxidation may trigger protein misfolding in sporadic amyloid diseases, which are clinically the most relevant neurological brain diseases, including Alzheimer's and Parkinson's diseases (Bieschke J. et al, Acc. Chem. Res. 2006; 39:611-619).

Not only adults are vulnerable to disorders mediated by oxidative stress. Neonates, including healthy neonates, are also subjected to damage caused by oxidative stress (Yasemin Ozsurekci, Kubra Aykac, “Oxidative Stress Related Diseases in Newborns”, Oxidative Medicine and Cellular Longevity, vol. 2016, Article ID 2768365, 9 pages, 2016). The main causes of oxidative stress in infants include birth itself, due to the transition from a hypoxic environment in the womb to a normoxic but relatively hyperoxic extrauterine environment, and a high metabolic rate requiring a high level of mitochondrial respiration and subsequent enhanced mitochondrial superoxide formation in an organism with a not yet fully developed antioxidant system (Friel et al. Evidence of Oxidative Stress in Full-Term Healthy Infants. Pediatric Research vol 56, no. 6, 2004).

Preterm infants are particularly susceptible to oxidative stress damage and oxidative stress-mediated disorders. Preterm infants with a gestational age of less than 30 weeks or a weight of less than 1500 g, have a major risk to develop these disorders. At cerebral level, there is a predisposition to oxidative stress damage due to the high amount of polyunsaturated fatty acids in the immature brain, particularly in neuronal membranes, but also due to the relatively high amount of protein-unbound iron. Immaturity associated with preterm birth and also oxygen therapy used for the treatment of respiratory distress significantly increase oxidative stress in preterm infants. Importantly, free radicals may damage the preterm brain. Consequently, to prevent long-term sequelae of oxidative stress, it is necessary to reduce and prevent oxidative stress damage (de Almeida, et al. Neonatal Diseases and Oxidative Stress in Premature Infants: An Integrative Review. J. Pediatr. 2021, 23). Oxidative stress-related neonatal disorders have also been described as “oxygen radical disease of neonatology” (Yasemin Ozsurekci, Kubra Aykac, “Oxidative Stress Related Diseases in Newborns”, Oxidative Medicine and Cellular Longevity, vol. 2016, Article ID 2768365, 9 pages, 2016).

Nutritional components have been suggested as antioxidants. Lutein, for instance, is a xanthophyll carotenoid known for scavenging reactive oxygen species thereby being suggested against oxidative stress-mediated disorders (Jin Ahn & Kim, Lutein as a Modulator of Oxidative Stress-Mediated Inflammatory Diseases. Antioxidants 2021, 10, 1448). Other studies have reported that casein phosphopeptides may act as antioxidants (Tenenbaum et al. Identification, production and bioactivity of casein phosphopeptides—A review, Food Research International vol. 157, July 2022), and that diets rich in fats comprising palmitic acid esterified to the sn-2 position have been described to increase enzymatic antioxidants (Lu et al. High Beta-Palmitate Fat Controls the Intestinal Inflammatory Response and Limits Intestinal Damage in Mucin Muc2 Deficient Mice. Plos One. June 2013, vol. 8, issue 6).

Recent research described a protective effect of early life supplementation with GOS against oxidative stress in piglets (Tian et al. Early-life galacto-oligosaccharides supplementation alleviates the small intestinal oxidative stress and dysfunction of lipopolysaccharide-challenged suckling piglets. Journal of Animal Science and Biotechnology (2022) 13:70).

Nevertheless, there remains a need for improved compositions for reducing or preventing oxidative stress damage and preventing oxidative stress-mediated disorders which employ safe components that may be used by both healthy and unhealthy human subjects ranging from infants to elderly.

### SUMMARY OF THE INVENTION

The present inventors have surprisingly found that a composition comprising lutein, short chain fatty acids (i.e., the fermentation products of non-digestible carbohydrates in the gastro-intestinal tract) and at least one of casein phosphopeptides or 1,3-dioleoyl-2-palmitoylglycerol synergistically reduce hydrogen peroxide induced apoptosis in gut epithelial cells. Without wishing to be bound by theory, the inventors hypothesize that said composition protects cells against hydrogen peroxide induced mitochondrial dysfunction and endoplasmic reticulum (ER) stress, thereby reducing DNA damage and, consequently, apoptosis. Oxidative stress damage and/or oxidative stress-mediated disorders are thereby reduced or prevented.

Accordingly, in a first aspect of the invention, the invention relates to a composition comprising lutein, non-digestible carbohydrates and at least one of casein phosphopeptides or 1,3-dioleoyl-2-palmitoylglycerol for use in reducing or preventing oxidative stress damage and/or preventing oxidative stress-mediated disorders in a human subject.

A second aspect of the invention relates to a composition comprising lutein, non-digestible carbohydrates and at least one of casein phosphopeptides or 1,3-dioleoyl-2-palmitoylglycerol for use in reducing oxidative stress-mediated gut barrier dysfunction in a human subject.

A third aspect of the invention relates to a nutritional composition comprising digestible carbohydrates, lipids, proteins and an energy content of at least 400 kcal per 100 g of the composition, wherein the composition comprises:
a. 60 to 430 μg lutein per 100 g of composition;
b. 1.5 to 7.5 wt. % non-digestible oligosaccharides, based on total composition, selected from galacto-oligosaccharides, fructo-oligosaccharides, human milk oligosaccharides or a combination thereof; and
c. 25 to 160 mg casein phosphopeptides per 100 g of composition, or 2 to 12 g 1,3-dioleoyl-2-palmitoylglycerol per 100 g composition.

Advantageously, the compositions may be used by a wide range of the population, including healthy and vulnerable infants as well as adults and elderly people since controlling oxidative stress damage may prevent a number of associated disorders.

### FIGURES

FIG. 1 shows the effect of SCFA's, lutein and CPP on H₂O₂-induced apoptosis (by measuring PS:Annexin V binding on the cell surface by luminescence and expressed as area under the curve).

FIG. 2 shows the effect of SCFA's, lutein and OPO on H₂O₂-induced apoptosis (by measuring PS:Annexin V binding on the cell surface by luminescence and expressed as area under the curve).

FIG. 3 shows the effect of SCFA's, lutein and CPP on H₂O₂-induced inflammation (by measuring IL-8 production).

FIG. 4 shows the effect of SCFA's, lutein and OPO on H₂O₂-induced inflammation (by measuring IL-8 production).

FIG. 5 shows the effect of SCFA's, lutein and CPP on H₂O₂-induced DNA damage (by measuring 8-OhdG production).

FIG. 6 shows the effect of SCFA's, lutein and OPO on H₂O₂-induced DNA damage (by measuring 8-OhdG production).

### DETAILED DESCRIPTION OF THE INVENTION

In a first aspect, the invention relates to a composition comprising lutein, non-digestible carbohydrates and at least one of casein phosphopeptides or 1,3-dioleoyl-2-palmitoylglycerol for use in reducing or preventing oxidative stress damage and/or preventing oxidative stress-mediated disorders in a human subject.

In some jurisdictions, the present invention may be defined as a method for reducing or preventing oxidative stress damage and/or preventing oxidative stress-mediated disorders in a human subject, the method comprising administering to said subject a composition comprising lutein, non-digestible carbohydrates and at least one of casein phosphopeptides or 1,3-dioleoyl-2-palmitoylglycerol.

In some jurisdictions, the present invention may be defined as the use of lutein, non-digestible carbohydrates and at least one of casein phosphopeptides or 1,3-dioleoyl-2-palmioylglycerol in the manufacture of a composition for reducing or preventing oxidative stress damage and/or preventing oxidative stress-mediated disorders in a human subject.

In a second aspect, the invention relates to a composition comprising lutein, non-digestible carbohydrates and at least one of casein phosphopeptides or 1,3-dioleoyl-2-palmitoylglycerol for use in reducing oxidative stress-mediated gut barrier dysfunction in a human subject.

In some jurisdictions, the invention may also be defined as a method for reducing oxidative stress-mediated gut barrier dysfunction in a human subject, the method comprising administering to said subject a composition comprising lutein, non-digestible carbohydrates and at least one of casein phosphopeptides or 1,3-dioleoyl-2-palmitoylglycerol.

In some jurisdictions, the invention may be defined as the use of lutein, non-digestible carbohydrates and at least one of casein phosphopeptides or 1,3-dioleoyl-2-palmioylglycerol in the manufacture of a composition for reducing oxidative stress-mediated gut barrier dysfunction in a human subject.

In some jurisdictions administering a nutritional composition to a human subject is considered non-therapeutic. In those instances the invention may be worded as defined above by way of a method comprising administering a nutritional composition. For clarity, the method can also be defined as a non-therapeutic method. By definition, the words “non-therapeutic” exclude any therapeutic effect.

The composition comprises lutein, non-digestible carbohydrates and at least one of casein phosphopeptides or 1,3-dioleoyl-2-palmitoylglycerol in a therapeutically efficient amount.

The term “composition”, as used herein refers to powder comprising at most 5 wt. % of water by weight of the powder. This powder may be suitably reconstituted with water or other food grade aqueous liquid, to form a ready-to drink composition in liquid form. Preferably 10-15 gram of this powder is added to 90 ml of liquid to arrive at a ready-to-drink liquid of 100 ml.

The term “oxidative stress”, as used herein, refers to a state of imbalance between oxidation and antioxidation in the body and is capable of causing damage to various cellular constituents, including DNA, and tissues leading to oxidative stress damage and, ultimately, oxidative stress-mediated disorders. Oxidative stress may result in, for example, cellular damage, impaired cell performance and/or cell death (“apoptosis”).

“Apoptosis” refers to the autonomous and orderly death of cells controlled by genes in order to maintain the stability of the internal environment. Different from cell necrosis, apoptosis is not a passive process, but an active process. It involves the activation, expression and regulation of a series of genes. Apoptosis is a basic biological phenomenon of cells and plays an essential role in the removal of unwanted or abnormal cells, or cells that have been damaged beyond repair in multicellular organisms. Apoptosis is a relevant marker of oxidative stress damage.

As used herein, “oxidative stress-mediated disorders” refers to conditions and diseases caused by the oxidative damage caused by imbalance between the oxidative and antioxidative systems in cells and tissues.

According to one preferred embodiment, “oxidative stress-mediated disorders” are selected from bacterial, viral, and parasitic infections, autoimmune disorders, cancer, atherogenic activity, kidney diseases, skin diseases, neurological and neurodegenerative diseases, age-related macular degeneration (AMD), cardiovascular diseases (CVD), enteric infections, gastritis, gastroesophageal reflux disease (GERD), chronic inflammatory diseases, particularly inflammatory bowel disease (IBD), irritable bowel syndrome (IBS), celiac disease, pancreatitis, hepatitis, sarcopenia, arthritis or diabetes. More preferably, the oxidative stress-mediated disorders are selected from cardiovascular diseases (CVD), neurological and neurodegenerative diseases, cancer, sarcopenia, and age-related macular degeneration (AMD).

In a preferred embodiment, the oxidative stress-mediated disorders are oxidative stress-mediated inflammatory disorders, more preferably oxidative stress-mediated inflammatory gastro-intestinal disorders and even more preferably oxidative stress-mediated inflammatory intestinal disorders. In a more preferred embodiment, the “oxidative stress-mediated disorders” are selected from inflammatory bowel disease, irritable bowel syndrome, enteric infections, gastroesophageal reflux disease (GERD) or gastritis.

According to a preferred embodiment, the oxidative stress-mediated disorders are oxygen radical diseases of neonatology, more preferably these oxygen radical diseases of neonatology are selected from bronchopulmonary dysplasia (BPD), retinopathy of prematurity (ROP), necrotizing enterocolitis (NEC), patent ductus arteriosus (PDA), periventricular leukomalacia (PVL), respiratory distress syndrome (RDS), intraventricular hemorrhage (IVH), periventricular leukomalacia (PVL), and brain damage (such as white matter lesions). Most preferably, the oxygen radical disease of neonatology is selected from necrotizing enterocolitis (NEC), respiratory distress syndrome (RDS) and brain damage (such as white matter lesions).

Oxidative stress damage may be measured in vivo by methods as described in Alzaid, F. et al., (2015). Biomarkers of Oxidative Stress in Blood. In: General Methods in Biomarker Research and their Applications. Biomarkers in Disease: Methods, Discoveries and Applications. Springer, Dordrecht. https://doi.org/10.1007/978-94-007-7696-8_41.

Preferably, the reduction of oxidative stress damage in a human subject is ≥3%, more preferably ≥5%, as determined by comparing the oxidative stress damage measured in a human subject administered the composition according to the invention, with the oxidative stress damage measured in a similar human subject not administered the composition according to the invention.

“Oxidative stress-mediated gut barrier dysfunction” refers to damages, lesions or malfunctioning of the gut barrier and gut barrier integrity caused by oxidative stress imbalance. Damage of gut epithelial cells is associated with gut barrier dysfunction which, in turn, facilitates infections by opportunistic pathogens. Damage of gut barrier is associated with ‘leaky gut’ diseases.

In a preferred embodiment, the human subject is selected from patients which have undergone abdominal surgery, patients that experience postoperative dysfunction of the gut and/or malnourished patients.

In another preferred embodiment, the human subject is an adult, preferably an adult of at least 50 years old, more preferably of at least 65 years old.

In a preferred embodiment, the composition for use in reducing or preventing oxidative stress damage and/or preventing oxidative stress-mediated disorders contributes to healthy aging.

In yet another preferred embodiment, the human subject is an infant or young child, preferably between 0-60 months of age, more preferably from 0 to 36 months of age.

Preferably, the human subject is an infant. The term “infant”, as used herein refers to a human subject with an age between 0 and 24 months, preferably between 0 and 12 months and even more preferably between 0 and 6 months.

The infant preferably is a preterm and/or small for gestational age (SGA) infant. A preterm infant relates to an infant born before the standard period of pregnancy is completed, i.e. before or on 37 weeks from the beginning of the last menstrual period of the mother. SGA infants refers to infants whose birth weight lies below the 10th percentile for their gestational age. They have usually been the subject of intrauterine growth restriction (IUGR). Preterm and/or SGA infants include low birth weight infants (LBW infants), very low birth weight infants (VLBW infants), and extremely low birth weight infants (ELBW infants). LBW infants are defined as infants with a weight less than 2500 g. VLBW infants as infants with a weight which is less than 1500 g, and ELBW infants as infants with a weight less than 1000 g.

### Lutein

As used herein, lutein is in the form of free xanthophylls, xanthophyll esters or other chemical forms of lutein. Lutein is found in high quantities in green leafy vegetables such as spinach, kale, and yellow carrots. Lutein may be obtained or isolated by any method recognized by those skilled in the art. For example, lutein may be obtained by extraction from marigolds or other xanthophylls-rich sources, chemical synthesis, fermentation or other biotechnology-derived and enriched xanthophyll sources. A suitable form of lutein useful in the present invention is available commercially as e.g. Floraglo® Lutein. The herein defined amounts of lutein refer to free lutein (i.e., equivalent to 100% pure lutein).

In a preferred embodiment, the composition comprises lutein in an amount ≥60 μg per 100 g of composition, preferably 65-430 μg per 100 g of composition, more preferably 70-400 μg per 100 g of composition, even more preferably 80-350 μg per 100 g of composition. Expressed differently, when the composition is reconstituted to a ready-to-drink composition in liquid form, the reconstituted composition preferably comprises lutein in an amount of 5-65 μg/100 ml, preferably 7-60 μg/100 ml, more preferably 8-55 μg/100 ml, even more preferably 8.5-50 μg/100 ml.

### Non-Digestible Carbohydrates

As used herein, the term “non-digestible carbohydrate” refers to oligosaccharides which are not digested in the intestine by the action of acids or digestive enzymes present in the human upper digestive tract, e.g. small intestine and stomach, but reach the distal portions of the intestines, such as the colon, intact where they are fermented by the human intestinal microbiota. For example, sucrose, lactose, maltose and maltodextrins are considered digestible saccharides.

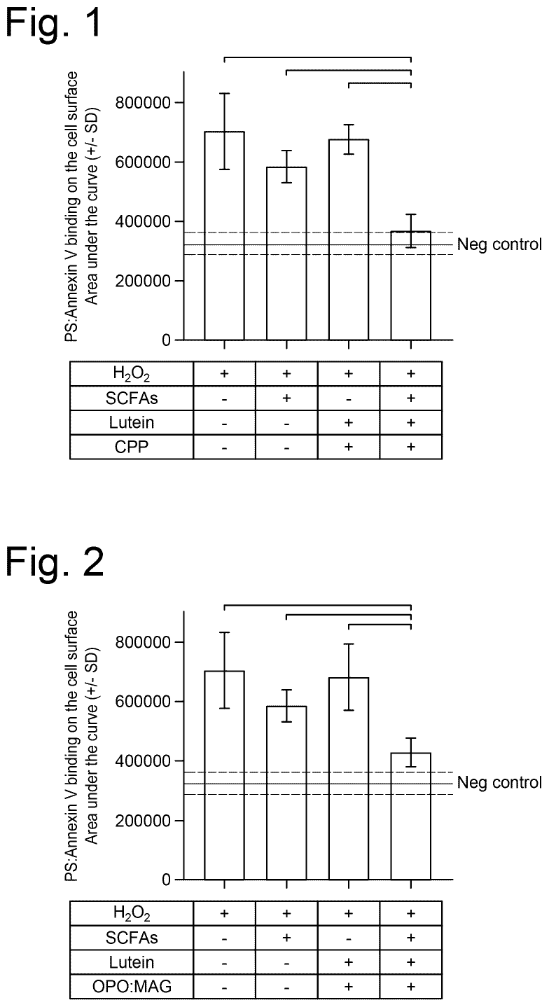

Microbial metabolites of non-digestible carbohydrates include short chain fatty acids (SCFA), for example acetate, propionate, butyrate, lactate, amongst others. The inventors have surprisingly found that the presence of the fermentation products of non-digestible carbohydrates, i.e. SCFA's, significantly reduce H₂O₂ induced apoptosis of gut epithelial cells when used in combination with lutein and at least one of casein phosphopeptides or 1,3-dioleoyl-2-palmitoylglycerol (see e.g. FIGS. 1 and 2).

Preferably, the non-digestible carbohydrates are non-digestible oligosaccharides, more preferably the non-digestible carbohydrates are selected from prebiotic oligosaccharides, human milk oligosaccharides (HMO), or combinations thereof.

The composition preferably comprises non-digestible carbohydrates in an amount of 0.25 wt. % to 25 wt. % non-digestible carbohydrates, more preferably 0.5 wt. % to 10 wt. %, even more preferably 1.5 wt. % to 7.5 wt. %, based on total composition. Expressed differently, when the composition is reconstituted to a ready-to-drink composition in liquid form, the reconstituted composition preferably comprises 80 mg to 4 g non-digestible carbohydrates per 100 ml, more preferably 150 mg to 2 g per 100 ml, even more preferably 300 mg to 1 g non-digestible carbohydrates per 100 ml.

### Prebiotic Oligosaccharides

The composition preferably comprises prebiotic oligosaccharides. Preferably, the composition comprises at least one prebiotic oligosaccharide, more preferably at least two prebiotic oligosaccharides.

Preferred prebiotic oligosaccharides have a DP in the range of 2 to 250, more preferably 2 to 60, most preferably below 40. Advantageously and most preferred, the prebiotic oligosaccharides are water-soluble (according to the method disclosed in L. Prosky et al, J. Assoc. Anal. Chem 71: 1017-1023, 1988).

Suitable prebiotic oligosaccharides are at least one, more preferably at least two, preferably at least three selected from the group consisting of fructo-oligosaccharides (FOS), galacto-oligosaccharides (GOS), xylo-oligosaccharides, arabino-oligosaccharides, arabinogalacto-oligosaccharides, gluco-oligosaccharides, chito-oligosaccharides, glucomanno-oligosaccharides, galactomanno-oligosaccharides, mannan-oligosaccharides, and uronic acid oligosaccharides.

More preferably, the prebiotic oligosaccharides selected from FOS, GOS, or mixtures thereof. More preferably, the composition comprises FOS and GOS at a weight ratio between (20 to 2):1, more preferably (16 to 4):1, even more preferably (12 to 6):1. Most preferably the weight ratio is about 9:1.

The GOS preferably are beta-galacto-oligosaccharides. Beta-galacto-oligosaccharides may also be referred to as trans-galacto-oligosaccharides (TOS). GOS is for example sold under the trademark Vivinal™ (Borculo Domo Ingredients, Netherlands) or Bi2Munno (Classado). Preferably the GOS comprises at least 80% beta-1,4 and beta-1,6 linkages based on total linkages, more preferably at least 90%. The GOS preferably has a DP of 2-60, more preferably a DP of 3-40, even more preferably a DP of 4-20 and most preferably a DP of 5-10.

FOS preferably comprises a chain of beta-linked fructose units with a DP of 2 to 250, more preferably 5 to 100, even more preferably 10 to 60. Preferably the FOS has an average DP above 20. FOS may includes inulin, levan and/or a mixed type of polyfructan. An especially preferred FOS is inulin. A FOS suitable for use in the compositions is also commercially available, e.g. Raftiline® HP (Orafti).

In one preferred embodiment, the non-digestible carbohydrates consist of prebiotic oligosaccharides and no human milk oligosaccharides.

The composition preferably comprises prebiotic oligosaccharides in an amount of 0.25 wt. % to 25 wt. %, more preferably 0.5 wt. % to 10 wt. %, most preferably 1.5 wt. % to 7.5 wt. %, based on total composition. Expressed differently, when the composition is reconstituted to a ready-to-drink composition in liquid form, the reconstituted composition preferably comprises 80 mg to 4 g prebiotic oligosaccharides per 100 ml, more preferably 150 mg to 2 g per 100 ml, even more preferably 300 mg to 1 g prebiotic oligosaccharides per 100 ml.

### Human Milk Oligosaccharides

The non-digestible carbohydrate preferably comprise human milk oligosaccharides.

“Human milk oligosaccharides” (HMO) are present in human milk and are non-digestible carbohydrates built from one or more of the following monomers: D-glucose, D-galactose, N-acetylglucosamine, L-fucose and sialic acid (N-acetylneuraminic acid).

Preferably, the HMO are selected sialyloligosaccharides, such as 3′-sialyllactose (3′-SL), 6′-sialyllactose (6′-SL), lactosialyl-tetrasaccharide a,b,c (LST), disialyllacto-N-tetraose (DSLNT), sialyllacto-N-hexaose (S-LNH), DS-LNH, and fucooligosaccharide, such as (un)sulphated fucoidan oligosaccharide, 2′-fucosyllactose (2′-FL), 3-fucosyllactose (3-FL), difucosyllactose, lacto-N-fucopenatose, (LNFP) I, II, III, IV Lacto-N-neofucopenaose (LNnFP), Lacto-N-difucosyl-hexaose (LNDH), and mixtures thereof.

In one preferred embodiment, the non-digestible carbohydrates consist of HMO and no prebiotic oligosaccharides.

The composition preferably comprises 0.038 wt. % to 12 wt. % HMO, preferably 0.075 wt. % to 9 wt. % HMO, more preferably 0.15 wt. % to 6 wt. % HMO, even more preferably 0.3 wt. % to 2.5 wt. % HMO. Expressed differently, when the composition is reconstituted to a ready-to-drink composition in liquid form, the reconstituted composition preferably comprises HMO in an amount of 0.01 g to 5 g per 100 ml, more preferably 0.05 g to 4.5 g per 100 ml of the composition, more preferably 0.1 g to 4.0 g per 100 ml of the composition, even more preferably 0.5 g to 3.5 g per 100 ml of the composition, most preferably 1 g to 3 g per 100 ml of the composition.

Preferably, the HMO is selected from 2′-fucosyllactose (2′-FL), 3-fucosyllactose (3-FL), 3-sialyllactose (3′-SL), 6-sialyllactose (6′-SL), lacto-N-tetrose (LNT), lacto-N-neotetrose (LNnT), or combinations thereof. More preferably, the HMO is selected from 2′-FL, 3-FL, 3′-SL and 6′-SL, or combinations thereof. Most preferably the HMO is 2′-FL.

2′-FL, preferably α-L-Fuc-(1→2)-β-D-Gal-(1→4)-D-Glc, is commercially available for instance from Sigma-Aldrich. Alternatively, it may be isolated from human milk, for example as described in Andersson & Donald, 1981, J Chromatogr. 211:170-1744, or produced by genetically modified micro-organisms, for example as described in Albermann et al, 2001, Carbohydrate Res. 334:97-103.

In yet another preferred embodiment, the non-digestible carbohydrates are a mixture of prebiotic oligosaccharides and HMO. More preferably, the non-digestible carbohydrates comprise GOS and FOS in combination with 2′-FL and/or LNT, most preferably GOS, FOS in combination with 2′-FL.

Preferably, the weight ratio of HMO (preferably 2′-FL) to prebiotic oligosaccharide (preferably, GOS) is from 5 to 0.05, more preferably 5 to 0.1, more preferably from 2 to 0.2. Preferably the weight ratio HMO (preferably 2′-FL) to prebiotic oligosaccharide (preferably FOS) is from 10 to 0.05, more preferably 5 to 0.1, more preferably from 2 to 0.5.

### Casein Phosphopeptides (CPP)

As used herein, CPP are defined as casein-derived peptides having at least one phosphoserine (SerP) residue per peptide molecule. CPP preferably comprises at least 1 SerP residue per 20 amino acids, more preferably at least 1 SerP residue per 10 amino acids or even 1-3 SerP residues per 7 amino acids. In addition to or instead of SerP, other phosphorylated amino acids, such as phosphothreonine (ThrP) or phosphotyrosine (TyrP) may be present. The CPP preferably has a phosphorus content between 0.6 and 1.5 wt. %. CPP may be prepared by enzymatic hydrolysis of casein or caseinate, especially whole casein, α-caseins, κ-casein or β-casein, for example using trypsin, pepsin, chymotrypsin, pancreatin or bacterial (Bacillus), fungal or plant endo- and/or exoproteases or mixtures thereof. Preferably the CPP is bovine CPP.

The composition preferably comprises CPP. Preferably, the composition comprises 25-160 mg, preferably 45-80 mg per 100 g of composition. Expressed differently, when the composition is reconstituted to a ready-to-drink composition in liquid form, the reconstituted composition preferably comprises 5-20 mg CPP per 100 ml, more preferably 6-16 mg per 100 ml of composition.

Preferably, the weight ratio of lutein to CPP ranges from 1:150 to 1:1000, more preferably from 1:200 to 1:900.

### 1,3-dioleoyl-2-palmitoylglycerol (OPO)

The composition preferably comprises 1,3-dioleoyl-2-palmitoylglycerol (OPO). Preferably, the composition comprises 2-12 g OPO per 100 g, more preferably 2.5-8 g OPO per 100 g composition. Expressed differently, when the composition is reconstituted to a ready-to-drink composition in liquid form, the reconstituted composition preferably comprises 0.27-1.62 g OPO per 100 ml, preferably 0.34-1.35 g OPO per 100 ml.

Suitable commercially available sources of OPO are e.g. from Loders Croklaan under the name Betapol™ or InFat™ of Enzymotec. Alternatively OPO may be prepared in a manner as for instance described in EP 0698078 and/or EP 0758846.

### Other Components

The composition is preferably an enteral nutritional composition, more preferably an oral nutritional composition. Preferably, the nutritional composition is a nutritionally complete composition. Non-limiting examples of compositions include fortified foods, supplements, nutraceuticals, capsules, powders, juices, milk powders, morning or evening supplements, and the like.

The composition preferably comprises digestible carbohydrates, lipids, proteins and combinations thereof.

Preferably, the composition comprises digestible carbohydrates. Preferred digestible carbohydrates are lactose, glucose, sucrose, fructose, galactose, maltose, starch and maltodextrin, more preferably the digestible carbohydrates are lactose. Preferably, the composition comprises at least 40 g digestible carbohydrates per 100 g of composition, more preferably 45-70 g digestible carbohydrates per 100 g composition. Expressed differently, when the composition is reconstituted to a ready-to-drink composition in liquid form, the reconstituted composition preferably comprises 5-9 g per 100 ml of digestible carbohydrates, preferably 6-8 g per 100 ml of digestible carbohydrates.

Preferably, the composition comprises protein. Preferred protein sources are dairy protein or plant protein. Preferably, the composition comprises 5-20 g protein per 100 g composition, more preferably 8-15 g protein per 100 g composition. Expressed differently, when the composition is reconstituted to a ready-to-drink composition in liquid form, the reconstituted composition preferably comprises 0.8-4 g protein per 100 ml, more preferably 1-3 g proteins per 100 ml of the ready-to-drink composition.

Preferably, the composition comprises lipid. Preferably, the composition comprises 15-35 g per 100 g composition, preferably 20-30 g per 100 g composition. Expressed differently, when the composition is reconstituted to a ready-to-drink composition in liquid form, the reconstituted composition preferably comprises 2.5-5 g lipids per 100 ml, preferably 3.0-4.5 g lipids per 100 ml.

Preferably, the lipid comprises linoleic acid and/or alpha-linolenic acid. Preferably, the composition comprises 1.5-6 g linoleic acid per 100 g composition. In another preferred embodiment, the composition comprises 150-550 mg alpha-linoleic acid per 100 g composition.

Preferably, the lipid comprises DHA and/or ARA. Preferably, the composition comprises 65-150 mg DHA per 100 g composition. Preferably, the composition comprises 100-200 mg ARA per 100 g composition.

In one preferred embodiment, the composition is selected from infant formula, follow on formula, growing up milk, human milk fortifier, supplements, baby food, weaning products, or the like. More preferably, the composition is an infant formula, follow-on formula, or young child formula. Even more preferably, the composition is an infant formula or follow-on formula, most preferably a infant formula or follow-on formula for a preterm infant or a small for gestational age infant.

In the present invention, infant formula refers to nutritional compositions, artificially made, intended for infants of 0 to about 4 to 6 months of age and are intended as a substitute for human milk. Typically, infant formulae are suitable to be used as sole source of nutrition. Such infant formulae are also known as starter formula. Follow-on formula for infants starting with at 4 to 6 months of life to 12 months of life are intended to be supplementary feedings for infants that start weaning on other foods. Infant formulae and follow-on formulae are subject to strict regulations, for example for the EU regulations no. 609/2013 and no. 2016/127. In the present context, young child formula refers to nutritional compositions, artificially made, intended for infants of 12 months to 36 months, which are intended to be supplementary feedings for infants. In the context of the present invention, young child formula may also be named growing-up milk.

In one preferred embodiment, the composition is an infant formula, follow-on formula or young child formula and preferably comprises:
3 to 7 g lipid/100 kcal, preferably 4 to 6 g lipid/100 kcal, most preferably 4.5 to 5.5 g lipid/100 kcal,
1.7 to 3.5 g protein/100 kcal, more preferably 1.8 to 3.0 g protein/100 kcal, most preferably 1.8 to 2.5 g protein/100 kcal; and
5 to 20 g digestible carbohydrates/100 kcal, more preferably 6 to 16 g digestible carbohydrates/100 kcal, most preferably 10 to 15 g digestible carbohydrate/100 kcal.

Preferably, the composition is an infant formula, follow-on formula or young child formula and has an energy density of 60 kcal to 75 kcal/100 ml, more preferably 60 to 70 kcal/100 ml, when in a ready-to-drink form.

The third aspect of the invention relates to a nutritional composition comprising digestible carbohydrates, lipids, proteins and an energy content of at least 400 kcal per 100 g of the composition, wherein the composition comprises:
a. 60 to 430 μg lutein per 100 g of composition;
b. 1.5 to 7.5 wt. % non-digestible oligosaccharides, based on total composition, selected from galacto-oligosaccharides, fructo-oligosaccharides, human milk oligosaccharides or a combination thereof; and
c. 25 to 160 mg casein phosphopeptides per 100 g of composition, or 2 to 12 g 1,3-dioleoyl-2-palmitoylglycerol per 100 g composition.

Preferably, the invention relates to a nutritional composition comprising digestible carbohydrates, proteins and lipids, wherein the composition is a powder composition comprising, per 100 g:
a. an energy content of at least 400 kcal;
b. 60-430 μg lutein;
c. 3.5-7.5 g non-digestible carbohydrates comprising fructo-oligosaccharides and galacto-oligosaccharides;
d. 45-70 g digestible carbohydrates, preferably lactose; the composition further comprising at least one of:
e. 25-80 mg casein phosphopeptides; or
f. 2-8 g 1,3-dioleoyl-2-palmitoylglycerol.

Preferably, all other optional ingredients described herein above for the uses and methods of the invention apply to the composition mutatis mutandis.

The present invention is illustrated by the following non-limiting examples.

### EXAMPLES

### Materials & Methods

### Ingredient Preparation

Concentrated stocks of each tested ingredient were prepared according to their solubility. Casein phosphopeptides (CPP, 100×) were prepared in 50 mM NaOH and used at a final concentration of 61.6 μg/mL. Short-chain fatty acids (75% sodium acetate/20% sodium propionate/5% sodium butyrate, B5887/P1880/S5636, Sigma-Aldrich) were resuspended in DMEM medium (31053028, Gibco) at a stock concentration of 4 M and applied at 2 mM final concentration. Micelles were used to deliver lutein into the cells. Lutein (PHR1699, Sigma-Aldrich) was prepared at a concentration of 1 mg/ml in 310 mM sodium taurocholate and 64.5 mM phosphatidylcholine. The final concentration applied to the cells was 0.415 μg/mL Lutein. A 2-palmitoylglycerol MAG (OPO) (75614, Sigma-Aldrich) concentrated stock solution was prepared at 100 mM in ethanol. The final concentration used in the experiments was 100 μM. Final concentrations of ingredients were obtained by dissolving the ingredient stock solutions in serum-free DMEM (Gibco™ 31053028) or PBS solution (PBS, 14190144, Gibco; 0.6% BSA; 1.2 mM CaCl₂; 1 mM MgCl₂).

### Cell Culture & Maintenance

A human Caco-2 cell line (HTB-37 ATCC) was used as a model of intestinal epithelium. Cells (passages 50-70) were maintained in a complete growth medium DMEM (High glucose+Glutamax, phenol red, 31966021, Gibco) supplemented with 10% heat-inactivated fetal bovine serum (10270106, Gibco), 1% penicillin-streptomycin (15140-130, Gibco), 1% non-essential amino acids in MEM (MEM NEAA 100×, 11140-035, Gibco), 1% sodium pyruvate in MEM (100 mM stock, 11360-039, Gibco). The cells were grown at 37° C. and 5% CO₂ and were passaged at 80% confluency.

### Apoptosis Assay

To study the impact of oxidative stress on apoptosis, Caco-2 cells were seeded at 1×10⁵ cells/mL in a white opaque 96-well plate (Corning Costar, 3917) (200 μL per well) and cultured for 4 days, until 100% confluency was reached. Then, cells were preincubated for 16 h at 37° C. 5% CO₂ with serum-deprived medium containing the ingredients. A negative control with medium without H₂O₂ was included. The tested ingredient(s) were tested in duplo per plate, with three plates per assay. Subsequently, cells were incubated for 1 h with 2 mM H₂O₂ in PBS solution (PBS, 14190144, Gibco; 0.6% BSA; 1.2 mM CaCl₂; 1 mM MgCl₂) with or without ingredients. Finally, the challenge medium was removed, and the cells were cultured in serum-deprived DMEM supplemented with or without the ingredients and the apoptosis reagent (from the Promega kit described below) for 21 h.

To determine apoptosis, the Real-Time Glo Annexin V Apoptosis and Necrosis Assay, JA1011, Promega was used. Luminescence (measuring PS:Annexin V binding on the cell surface as a marker for apoptosis) was measured at the beginning of the recovery and at regular intervals over the course of 21 h.

### Measurement of IL-8 and DNA Damage (8-OhdG)

To study the effects of the ingredients in a context of oxidative stress, markers of inflammation (IL-8) and DNA damage (8-OhdG), were measured using Caco-2 cells treated with hydrogen peroxide. Briefly, Caco-2 cells were seeded at 2.5×10⁵ cells/mL on 24-well plates (3526, Corning COSTAR®, Corning Inc.) and were grown for 23 days. Medium was refreshed every 2 to 3 days. On day 23, the medium in each well was removed and replaced with the ingredient-DMEM solutions (1 ml per well) for 16 h at 37° C. 5% CO₂. A negative control with medium without H₂O₂ was included. The tested ingredient(s) were tested in duplo per plate, with three plates per assay. Thereafter, the ingredient-DMEM solutions were removed, and the cells were challenged for 1 h with 1 mM hydrogen peroxide (H₂O₂) in PBS solution with or without the ingredient(s). After 1 h, the challenge medium was removed from the cells and the ingredients in PBS solution were added and incubated for 4 h. Following incubation, supernatants were collected, centrifuged at 2500 rpm for 10 min at 4° C. and used immediately for measuring either IL-8 or 8-OhdG with an ELISA kit.

A human IL-8 ELISA kit (RAB0319, Sigma-Aldrich) was used to determine IL-8 concentration. A human 8-OhdG ELISA kit (ab201734, Abcam) was used to assess DNA damage. Each commercial kit was used following the manufacturer's protocol. Luminescence measurements were performed by using a microplate reader (FlexStation 3, Molecular Devices).

### Statistical Analyses

Values are expressed as mean±SD. The number of experimental replicates was 3 replicates. Statistical analyses were performed using GraphPad Prism 9. For parametric data, possible differences were assessed with Two-Way ANOVA followed by LSD post-test. Differences with a p value lower than 0.05 were considered statistically significant.

### Results

### Results Apoptosis Assay

In FIG. 1 the effect of SCFA's, lutein and CPP on H₂O₂-induced apoptosis are shown. In FIG. 2 the effect of SCFA's, lutein and OPO on H₂O₂-induced apoptosis are shown. The area under the curve is determined from timepoints 0 to 21 h. Horizontal bars indicate significant differences between compared groups.

The combination of SCFA's, lutein with either CPP or OPO reduces the H₂O₂-induced apoptosis synergistically compared to the effect of SCFA's alone or the effect of lutein with either CPP or OPO on H₂O₂-induced apoptosis.

### Results IL-8 Assay

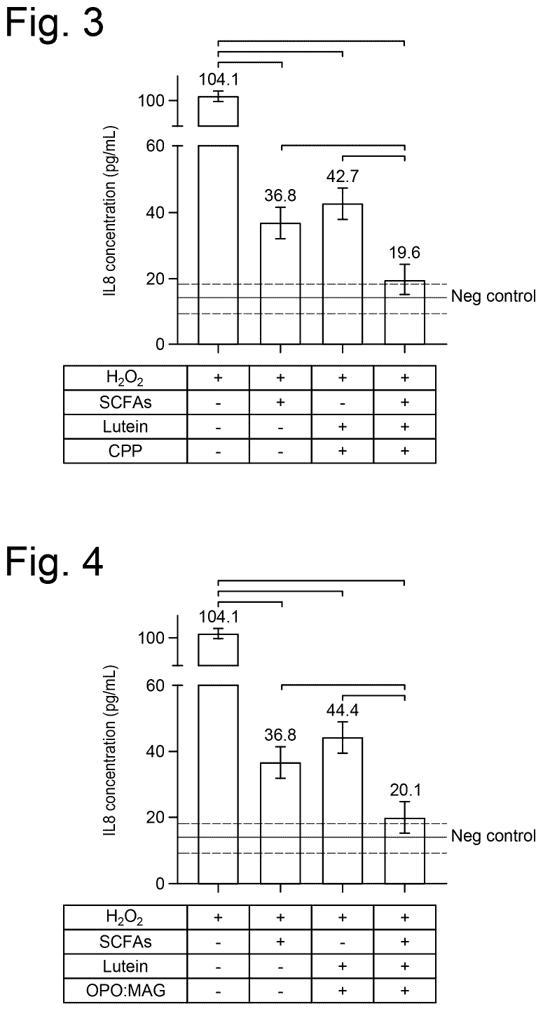

In FIG. 3 the effect of SCFA's, lutein and CPP on H₂O₂-induced IL-8 production are shown. In FIG. 4 the effect of SCFA's, lutein and OPO on H₂O₂-induced IL-8 production are shown. Horizontal bars indicate significant differences between compared groups.

The combination of SCFA's, lutein with either CPP or OPO reduces significantly the amount of IL-8 produced compared to the effect of SCFA's alone or the effect of lutein with either CPP or OPO on IL-8 production.

### Results DNA Damage (8-OhdG)

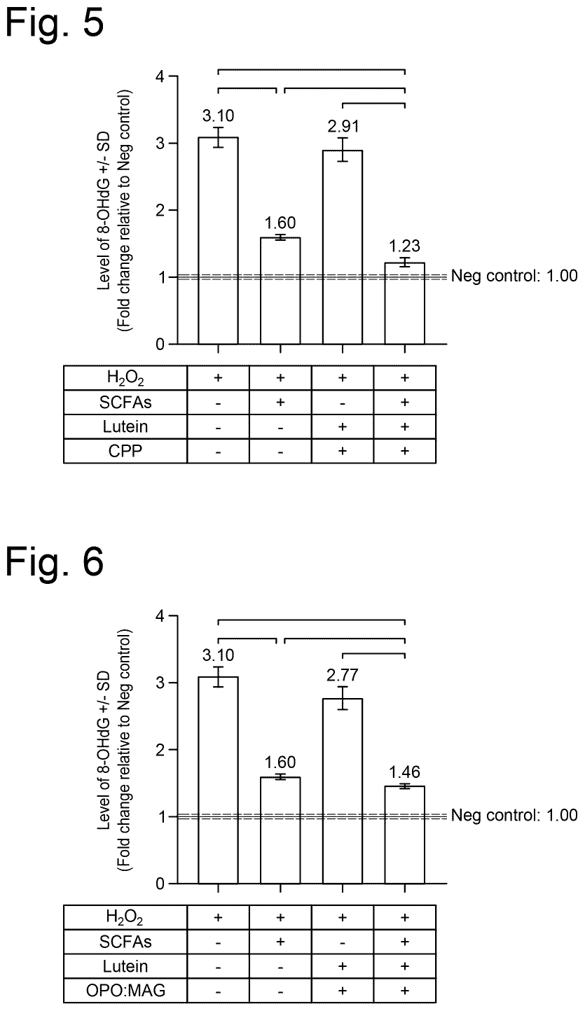

In FIG. 5 the effect of SCFA's, lutein and CPP on H₂O₂-induced 8-OhdG production are shown. In FIG. 6 the effect of SCFA's, lutein and OPO on H₂O₂-induced 8-OhdG production are shown. Horizontal bars indicate significant differences between compared groups.

The combination of SCFA's, lutein with either CPP or OPO reduces significantly the amount of 8-OhdG produced compared to the effect of SCFA's alone or the effect of lutein with either CPP or OPO on 8-OhdG production.

1. A composition comprising lutein, non-digestible carbohydrates and at least one of casein phosphopeptides or 1,3-dioleoyl-2-palmitoylglycerol for use in reducing or preventing oxidative stress damage and/or preventing oxidative stress-mediated disorders in a human subject.

2. A composition comprising lutein, non-digestible carbohydrates and at least one of casein phosphopeptides or 1,3-dioleoyl-2-palmitoylglycerol for use in reducing oxidative stress-mediated gut barrier dysfunction in a human subject.

3. The composition for use according to any of the preceding claims, wherein the non-digestible carbohydrates are non-digestible oligosaccharides, preferably the non-digestible carbohydrates are selected from prebiotic oligosaccharides, human milk oligosaccharides (HMO), or combinations thereof.

4. The composition for use according to any of the preceding claims, wherein the non-digestible carbohydrates are prebiotic oligosaccharides, preferably prebiotic oligosaccharides selected from fructo-oligosaccharides (FOS), galacto-oligosaccharides (GOS), or mixtures thereof.

5. The composition for use according to any of the preceding claims, wherein the non-digestible carbohydrates are HMO, preferably HMO selected from 2′-fucosyllactose (2′-FL), 3-fucosyllactose (3-FL), 3′-sialyllactose (3′-SL), 6′-sialyllactose (6′-SL), lacto-N-tetrose (LNT), lacto-N-neotetrose (LNnT), or combinations thereof.

6. The composition for use according to any of the preceding claims, wherein the composition comprises 25-160 mg casein phosphopeptides per 100 g of composition.

7. The composition for use according to any of the preceding claims, wherein the composition comprises 2 to 12 g of 1,3-dioleoyl-2-palmitoylglycerol per 100 g composition.

8. The composition for use according to any of the preceding claims, wherein the reduction of oxidative stress damage in a human subject is ≥3%, preferably ≥5%, as determined by comparing the oxidative stress damage measured in a human subject administered the composition according to any of the preceding claims with the oxidative stress damage measured in a similar human subject not administered the composition according to any of the preceding claims.

9. The composition for use according to any of the preceding claims, wherein oxidative stress-mediated disorders are oxidative stress-mediated inflammatory disorders, preferably oxidative stress-mediated inflammatory gastro-intestinal disorders.

10. The composition for use according to claim 9, wherein oxidative stress-mediated inflammatory gastro-intestinal disorders are selected from inflammatory bowel disease, irritable bowel syndrome, enteric infections, gastroesophageal reflux disease (GERD) or gastritis.

11. The composition for use according to any of the preceding claims, wherein the human subject is an adult, preferably an adult of at least 50 years old.

12. The composition for use according to claim 11, wherein the use contributes to healthy aging.

13. The composition for use according to claim 11 or 12, wherein oxidative stress-mediated disorders are selected from cardiovascular diseases (CVD), neurological and neurodegenerative diseases, cancer, sarcopenia, and age-related macular degeneration (AMD).

14. The composition for use according to any of claims 1 to 10, wherein the human subject is an infant or young child, preferably between 0-60 months of age.

15. The composition for use according to claim 14, wherein the infant is a preterm infant, and/or a small for gestational age infant.

16. The composition for use according to claim 14 or 15, wherein the oxidative stress-mediated disorders are selected from necrotizing enterocolitis (NEC), respiratory distress syndrome (RDS) and brain damage.

17. A nutritional composition comprising digestible carbohydrates, lipids, proteins and an energy content of at least 400 kcal per 100 g of the composition, wherein the composition comprises:
a. 60 to 430 μg lutein per 100 g of composition;
b. 1.5 to 7.5 wt. % non-digestible oligosaccharides, based on total composition, selected from galacto-oligosaccharides, fructo-oligosaccharides, human milk oligosaccharides or a combination thereof; and
c. 25 to 160 mg casein phosphopeptides per 100 g of composition, or 2 to 12 g 1,3-dioleoyl-2-palmitoylglycerol per 100 g composition.
